(19) United States
(12) Patent Application Publication
(10) Pub. No.: US 2013/0034464 A1
(43) Pub. Date: Feb. 7, 2013

(54) Active decontamination microcapsule filtration media

(71) Applicant: Jesse W. Taylor, Baldwinsville, NY (US)

(72) Inventor: Jesse W. Taylor, Baldwinsville, NY (US)

(73) Assignee: SRC, INC., North Syracuse, NY (US)

(21) Appl. No.: 13/558,898

(22) Filed: Jul. 26, 2012

Related U.S. Application Data
(60) Provisional application No. 61/514,653, filed on Aug. 3, 2011.

(51) Int. Cl. A62D 3/30; B01D 53/30; C02F 1/50; C02F 1/58; A61L 9/00; B01J 19/00

(52) U.S. Cl. 422/3; 422/108; 210/739; 210/96.1; 95/8; 96/226; 96/417

(57) ABSTRACT

Filtration devices, methods, and systems comprising a decontamination filtration medium. The filtration medium comprises a remotely-triggered decontamination material stored in microcapsules that are embedded in or attached to a filter element. The microcapsules contain a decontamination agent that will detoxify one or more toxins, including but not limited to chemical and biological warfare agents, and industrial materials and chemicals, among other things. The microcapsules can be locally or remotely triggered to release the detoxifying agent by a triggering agent such as light, electrical potential, pressure, temperature, or other methods. One method of use of the filtration device comprises the following: when a target toxin is detected in the vicinity of the device, the microcapsules are actively triggered to release the detoxifying agent, thereby neutralizing the toxic agent.

### CROSS-REFERENCE TO RELATED APPLICATIONS

This application claims priority to U.S. Provisional Application No. 61/514,653, filed on Aug. 3, 2011 and entitled “Active Decontamination Microcapsule Filtration Media,” which is hereby incorporated by reference in its entirety.

### BACKGROUND OF THE INVENTION

1. Field of the Invention

The present invention relates to a filtration device and, more specifically, to a filtration device comprising an active decontamination filtration medium for remediation of chemical and biological warfare agents, toxic chemicals and materials, and/or industrial chemicals and materials.

2. Description of the Related Art

The state-of-the-art in filtration devices for contaminant removal is devices which contain activated carbon. While activated carbon technology is effective, it typically requires a large bed of adsorption media. These large adsorption bed volumes require large amounts of space, and often result in significant pressure drops across the filter bed. A large pressure drop across the filter bed, in turn, can significantly impact the capital cost to build the system as well as the operating costs due to required over-sizing of many components. Also, in an activated carbon system there is at most only minimal detoxification of air, with mostly just the removal of offending species from the air. The toxic species remain toxic and the contaminated activated carbon bed becomes toxic itself, thereby requiring remediation, including specialized and expensive waste and detoxification expenses.

Innovative technologies are therefore required for decontaminating filtration devices which capture and inactivate contaminates and thus protect people from accidental or malicious release of harmful materials.

### BRIEF SUMMARY OF THE INVENTION

It is therefore a principal object and advantage of the present invention to provide a filtration device that comprises active decontamination during filtration.

It is yet another object and advantage of the present invention to provide a filtration device that comprises an active decontamination filtration medium.

It is another object and advantage of the present invention to provide a filtration device with a highly reactive decontamination medium that provides a timely and effective response to exposure from a wide variety of contaminates including but not limited to toxic industrial chemicals (“TIC”), toxic industrial materials (“TIM”), and/or chemical and biological warfare agents (“CBA”).

It is a further object and advantage of the present invention to provide a filtration device comprising a filtration medium that comprises a microcapsule material.

It is another object and advantage of the present invention to provide a filtration device with microcapsules that can be triggered to release a detoxifying agent by a triggering agent such as light, electrical potential, pressure, temperature, or other activators.

Other objects and advantages of the present invention will in part be obvious, and in part appear hereinafter.

In accordance with the foregoing objects and advantages, the present invention provides a decontamination device comprising a filtration medium, the medium comprising one or more microcapsules. The microcapsules comprise a decontamination agent responsive to one or more contaminants. The decontamination agent is any agent capable of neutralizing or trapping a contaminant, including but not limited to Spilfyter® Decontamination Solution 2, Supertropical bleach, EasyDecon®, QAC Decontamination Solution, M100 Sorbent Decontamination, Dahlgren Green, NaOCl, LiOCl, Ca(OCl)₂, bleach, hydrogen peroxide, peracetic acid, and/or L-Gel, among many other known decontamination and/or trapping agents. The microcapsule can comprise two or more decontamination agents.

In one aspect of the invention, the microcapsule is triggered to release the encapsulated decontamination agent by a local or remote trigger, such as a sensor that detects the target causes the activation and/or release of the decontamination agent from the microcapsule(s). In another aspect of the invention, a remote trigger such as light, electrical impulse, pressure, temperature, or a variety of other methods located remote from the microcapsules cause the microcapsules to release the decontamination agent.

The present invention also provides a method of use for the filtration device and medium comprising the steps: (i) detecting a contaminant either at the microcapsule level or remotely; (ii) releasing the encapsulated decontamination agent; and (iii) contacting the contaminant with the decontamination agent. In one aspect of the invention, the contaminant is detected and a remote trigger is activated, which in turn activates a triggerable agent in the microcapsules, thereby causing release of the encapsulated decontamination agent. For example, the method can comprise the steps of: (i) detecting a contaminant; (ii) activating the remote trigger; (iii) causing activation of the triggerable agent by the remote trigger; (iv) causing the microcapsule to release the decontamination agent by the triggerable agent (e.g., causing the microcapsule to explode or otherwise crack, dissolve, or expose its interior components to the exterior environment); and (v) contacting the contaminant with the decontamination agent to deactivate, encapsulate, or otherwise neutralize the contaminant.

According to an aspect of the invention is provided, therefore, a method for neutralizing a target pathogen or substance, comprising the steps of: (i) exposing a volume of water or air to a filtration device, the filtration device comprising: a filtration medium comprising a plurality of microcapsules, each of the microcapsules containing a neutralizing agent, wherein the neutralizing agent is released from at least one of the plurality of microcapsules in response to an activation signal; (ii) detecting the target pathogen or substance; (iii) initiating an activation signal upon detection of the target pathogen or substance; (iv) releasing the neutralizing agent from at least one of the plurality of microcapsules in response to the activation signal; and (v) neutralizing the target pathogen or substance with the released neutralizing agent. The microcapsules can comprise two or more different neutralizing agents, with a first plurality of the microcapsules comprise a first neutralizing agent, and a different plurality of the microcapsules comprise a second neutralizing agent. Once the air or water is filtered, the resulting neutralized volume of water or air can be released from the filtration device. According to another embodiment, the filtration device comprises a second filtration medium, with the second filtration medium adapted to filter the neutralized target pathogen or substance from the water or air.

According to a second aspect of the invention is provided a system for neutralizing a target pathogen or substance comprising: (i) a filtration device, the filtration device comprising a filtration medium comprising a plurality of microcapsules, each of said microcapsules containing a neutralizing agent; and (ii) a sensor adapted to detect the target pathogen or substance and, upon detection of the target pathogen or substance, send an activation signal to the plurality of microcapsules; (iii) wherein said at least one of said plurality of microcapsules is adapted to release said neutralizing agent in response to the activation signal. The system can comprise two or more different neutralizing agents, with a first plurality of the microcapsules comprise a first neutralizing agent, and a different plurality of the microcapsules comprise a second neutralizing agent. Once the air or water is filtered, the resulting neutralized volume of water or air can be released from the filtration device. According to another embodiment, the filtration device comprises a second filtration medium, with the second filtration medium adapted to filter the neutralized target pathogen or substance from the water or air.

According to a third aspect of the invention is provided a method for neutralizing a target pathogen or substance using a neutralizing agent, comprising the steps of: (i) exposing a volume of water or air to a filtration device, the filtration device comprising: (a) a sensor adapted to detect the target pathogen or substance; and (b) a filtration medium comprising a plurality of microcapsules, wherein a first group of the plurality of microcapsules comprise a first neutralizing agent precursor and a second group of the plurality of microcapsules comprise a second neutralizing agent precursor, and further wherein the first and second neutralizing agent precursors are released from the plurality of microcapsules in response to an activation signal; (ii) detecting, by the sensor, the target pathogen or substance; (iii) initiating an activation signal upon detection of the target pathogen or substance; (iv) releasing the first and second neutralizing agent precursors from the plurality of microcapsules in response to the activation signal, wherein the released first and second neutralizing agent precursors interact to create the neutralizing agent; (v) neutralizing the target pathogen or substance with the neutralizing agent; and (vi) releasing the resulting neutralized volume of water or air from the filtration device.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of a filter comprising an active microcapsule decontamination medium according to an aspect of the invention;

FIG. 2 is a flowchart of a method for filtration according to an aspect of the invention; and

FIG. 3 is a schematic of a filter comprising an active microcapsule decontamination medium according to an aspect of the invention.

### DETAILED DESCRIPTION OF THE INVENTION

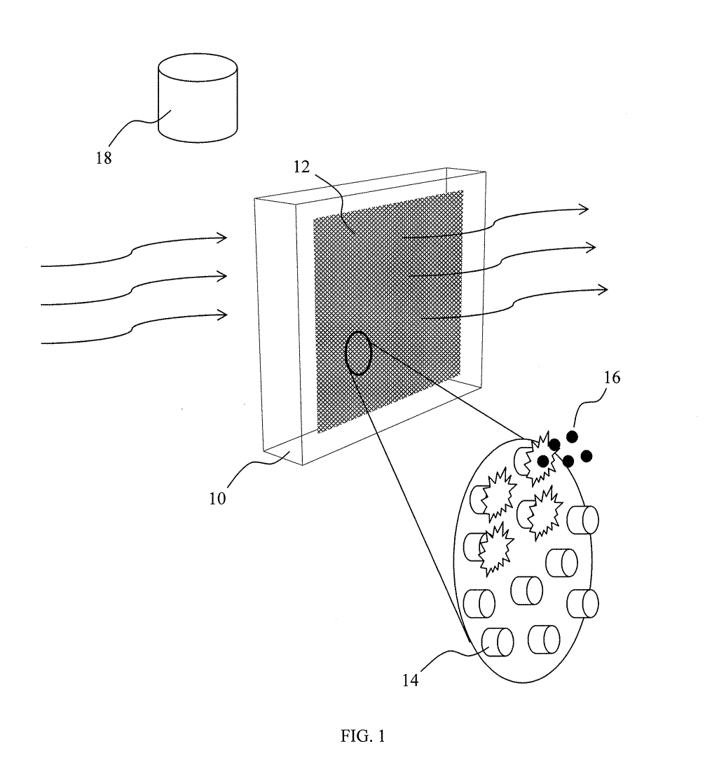

Referring now to the drawings, wherein like reference numerals refer to like parts throughout, there is seen in FIG. 1 a filtration device 10 which contains an active decontamination filtration medium 12. The filtration device can be adapted to target a wide variety of target agents, including but not limited to chemical agents such as sarin and other nerve agents and mustard gas; bacterial pathogens such as Anthrax, Brucellosis, and Plague; viral pathogens such as smallpox and hemorrhagic fevers; biological toxins such as ricin and botulinum; and novel threat agents, among many, many others.

Although filtration device 10 is depicted as a box-like structure in FIG. 1, it should be understood that filtration medium 12 can be configured or adapted for any use, regardless of the size or shape of the area to be filled with the medium. The size and shape of filtration medium 12 is limited only by the manufacturer's ability to create the desired size or shape. For example, the filtration media can be a stand-alone component or it can be inserted or manufactured into a frame or other supporting structure. It can be inserted into a small space, such as a portion of a breathing device, or can comprise a large surface area, such as an air intake.

As just a few of many non-limiting examples of potential applications, formulations, and/or embodiments of the filtration device, the filter device or the filtration medium can be used in or on ships, buildings, subway systems, forward command centers, tanks, or any facility comprising high-value assets, among others. For example, an embodiment of the device or method could be established at all air-intakes for any of the above, thereby protecting the interior of the structure—and any inhabitants therein—from target agents or pathogens.

Filtration medium 12 can comprise one or more microcapsules 14, which can be embedded in or on the filtration medium, or added to the interior or exterior of the medium. In one embodiment, the microcapsules can be located only on the inside or outside of the filtration medium, for example. In another embodiment, the microcapsules can comprise a medium located between or outside of the filtration medium, forming layers of filtration medium. The microcapsules can be formed of, for example, organic materials, such as polymeric species, or inorganic materials, such as oxides, although this list is not meant to be comprehensive. There are a wide variety of microencapsulation methods and techniques, and any one of these methods may be used to create, or encapsulate material to form, microcapsules 14. Further, each of the microcapsules in a filtration device may be manufactured from the same material, or a filtration device may represent two or more varieties of microcapsule material.

As just a few of many non-limiting examples of potential applications, formulations, and/or embodiments of the microcapsules according to the filtration device or method are microcapsules comprising one or more of the following: polystyrene, polyamides, polyurethanes, polyureas, and/or other suitable microcapsule material, including material already known in the art to function or capable of functioning as a (preferably reversible) encapsulating material.

Microcapsules 14 encapsulate a decontamination agent 16 initially located inside each microcapsule. Decontamination agent 16 is, for example, inserted inside the formed microcapsule, or the microcapsule can be formed around the decontamination agent. According to one embodiment, decontamination agent 16 is a highly reactive compound that quickly and effectively neutralizes a target toxin, contaminant, or other molecule or agent. According to one embodiment, decontamination agent 16 is chosen from, but not limited to, one or more commercial products and/or a combination of products such as Spilfyter® Decontamination Solution 2, Supertropical bleach, EasyDecon®, QAC Decontamination Solution, M100 Sorbent Decontamination, Dahlgren Green, NaOCl, LiOCl, Ca(OCl)₂, bleach, hydrogen peroxide, peracetic acid, and/or L-Gel, among many other known decontamination and/or trapping agents. According to another embodiment, decontamination agent 16 is a commercial product, or a proprietary product or mixture. According to yet another embodiment, the decontamination agent located inside a single microcapsule 14 is a mixture of two or more agents targeting one or more toxic compounds or materials.

According to one embodiment, decontamination agent 16 initially comprises two or more reagents, each initially stored separately inside separate microcapsules. When the system is triggered the two or more separate microcapsules release the two or more precursor reagents which then combine or react to form decontamination agent 16. For example, a first sub-population of the microcapsules store reagent X and a second, separate sub-population of the microcapsules store reagent Y (and so on). When the system is triggered, both sub-populations release reagents more or less at the same time. Just-released reagent X and reagent Y then combine, react, or otherwise interact to result in decontamination agent Z which is free to interact with the detected target. This setup would be beneficial in numerous situations including, for example, in circumstances where decontamination agent Z is only stable for a short period of time.

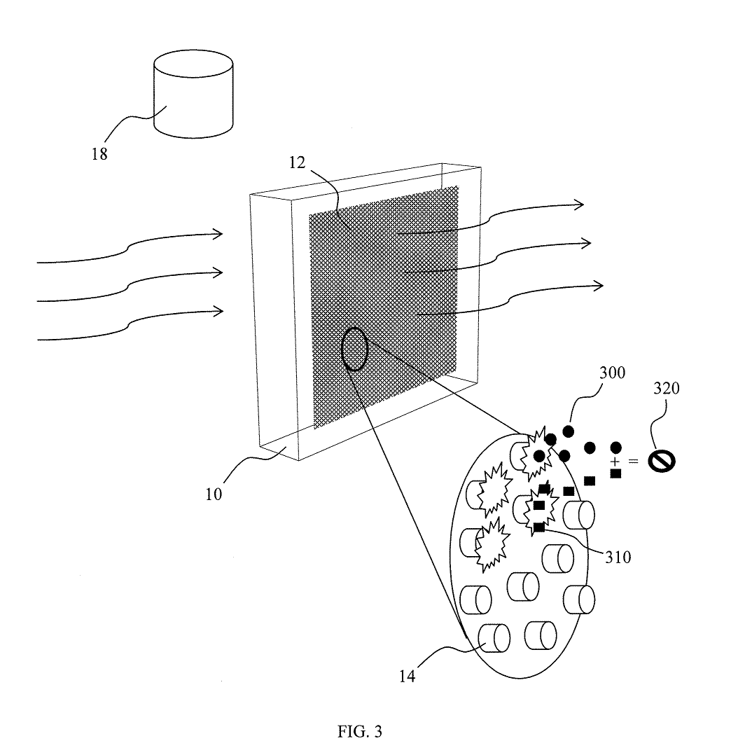

An embodiment of this configuration is depicted in FIG. 3. Instead of a fully-active decontamination agent, microcapsules 14 contain just one of two or more decontamination agent precursors which must combine after release in order to form the active agent. In the figure, decontamination agent precursor 300 is stored in one subset of microcapsules 14, and decontamination agent precursor 310 is stored in a second, separate, subset of microcapsules 14. When decontamination agent precursors 300 and 310 are released in response to a trigger, they can interact (including but not limited both covalent and non-covalent interactions or reactions) to form an active decontamination agent 320 which is then free to neutralize the target.

According to one embodiment, decontamination agent 16 responds to a target contaminating agent by detecting that agent in its proximity. For example, microcapsule 14, filtration medium 12, or filter device 10 can comprise one or more sensors that detect the target agent/compound/material/toxin and cause the activation and/or release of decontamination agent 16 from microcapsule 14.

As just a few of many non-limiting examples of potential applications, formulations, and/or embodiments of a target sensor, detection, or analytic system according to the filtration device or method are one or more commercial and/or proprietary sensor systems such as those manufactured or sold by Proengin®, FLIR®, Bertin Technologies®, Bruker®, Drager®, and/or Thermo Scientific®, among many others.

Microcapsules 14 can also comprise a remotely triggerable agent which is triggered by a remote trigger agent 18. For example, the remotely triggerable agent can comprise, among other things, carbon nanotubes, photosensitive organic molecules, metal oxides and/or metal particles. Accordingly, the remotely triggerable agent can be activated by a remote trigger agent such as light, electrical potential, pressure, temperature, magnetic, or other methods sufficient to induce the release of decontamination agent 16.

There are numerous uses and methods of use for filtration device 10. For example, according to one method, one or more decontamination agents are encapsulated into microcapsules forming part or all of the filtration device. The triggerable active decontamination filtration system can be targeted at one or more TICs, TIMs, and/or CBAs. When a TIC, TIM, or CBA is detected either by the filtration device or in the vicinity of the device, or a contaminant is merely suspected, a light 18 activates in the vicinity of the device. The causes the release of the detoxifying agent into the filter element allowing for immediate decontamination of, for example, airborne materials. The light—and the response of the triggerable agent—can comprise a tuned frequency such that sunlight alone does not activate the triggerable agent.

A triggerable decontamination device provides numerous benefits, including minimization of the spread of the harmful materials and decreased contamination of any HVAC system where the device is installed. These novel systems will allow for significantly improved reaction time to a potential release of harmful materials thereby saving lives and decreasing total remediation time, effort, and costs.

The present device or system can comprise embodiments in which the device or system include or function in conjunction with existing filtration systems, devices, or methods. For example, the novel filtration system described herein and the traditional filtration system can operate in series or in parallel. According to one embodiment, filtration device 10 is located in the flow of air downstream of a traditional filter or system such that the device will only activate if a target or pathogen successfully penetrates the traditional filter. Alternatively, if sensitivity or detection of the target is vital, filtration device 10 can be located in the flow of air upstream of the traditional filter. A system comprising both the novel filtration system or method together with existing filtration systems or methods could improve (or modulate as desired) sensitivity, extend the lifetime of one or both filtration systems, reduce logistics, decrease the footprint of the systems, and provide numerous other benefits.

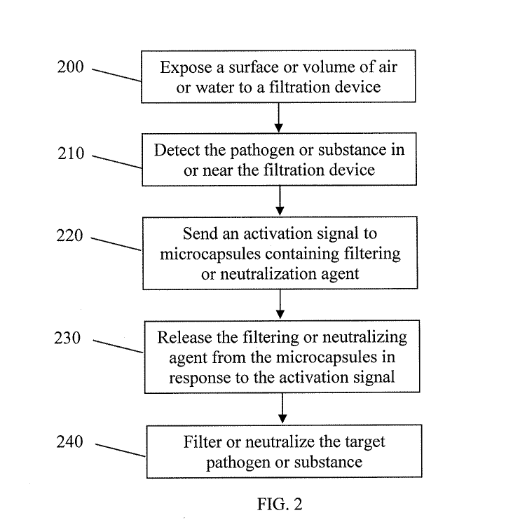

FIG. 2 depicts a method of filtration according to one embodiment of the invention. At step 200, a surface, volume of air, and/or volume of water is exposed to any variant of the filtration device as described herein, including but not limited to the embodiment depicted in FIG. 1. For example, the air or water can be blown, pumped, or otherwise forced into, onto, or across the filtration medium. The filtration device comprises a plurality of microcapsules which encapsulate one or more different decontamination agents which are initially located inside each microcapsule. The decontamination agent is, for example, inserted inside the formed microcapsule, or the microcapsule can be formed around the decontamination agent. According to one embodiment, the decontamination agent is a highly reactive compound that quickly and effectively neutralizes a target toxin, contaminant, or other molecule or agent. According to one embodiment, the decontamination agent is chosen from, but not limited to, one or more commercial products and/or a combination of products such as Spilfyter® Decontamination Solution 2, Supertropical bleach, EasyDecon®, QAC Decontamination Solution, M100 Sorbent Decontamination, Dahlgren Green, NaOCl, LiOCl, Ca(OCl)₂, bleach, hydrogen peroxide, peracetic acid, and/or L-Gel, among many other known or novel decontamination and/or trapping agents. According to one embodiment, the filtration medium comprises a variety of different decontamination agents.

At step 210, the target pathogen, substance, TIC, TIM, and/or CBA is detected by a sensor, which is a component of the neutralization system. Sensors designed or otherwise able to detect or predict the presence of a target pathogen, substance, TIC, TIM, and/or CBA are well-known in the art and will not be described here. The sensor can be, but need not be, a component of the filtration device itself, and can be, but need not be, a component of the filtration medium. For example, the sensor can be located at a point in the air or water flow ‘upstream’ of the flow such that the sensor can sample the air or water before it reaches the filtration device. Alternatively, the sensor can be embedded in or otherwise attached to the filtration medium. In yet another embodiment, the sensor is a sensor array capable of detecting two or more target pathogens, substances, TICs, TIMs, and/or CBAs. As an example, the sensor can be an optical sensor, a spectroscopy method such as mass spectrometry or others, a MEMS sensor, a DNA or protein sensor, a conductivity or polarity sensor, or any other type of sensor capable of detecting one or more of the targets of interest.

At step 220, the sensor sends an activation signal to one or more of the microcapsules upon detection of a target toxin or substance. As just one example, the filtration system can comprise a MEMS sensor that sends an electrical signal when it detects ricin on a surface or in air or water. The electrical charge either directly causes microcapsules containing a ricin-neutralization or—filtration agent, such as neutralizing antibodies, to rupture or otherwise release the agent, or the electrical charge is converted into a different signal that ultimately causes the agent to be released from the microcapsules. According to one embodiment, the system is designed to only send an activation signal to those microcapsules that contain an agent specific to the detected target. At step 230, the agent is released from the microcapsules in response to the signal. Finally, at step 240, the target pathogen or substance is neutralized or filtered in response to release of the neutralization/filtration agent from the microcapsules.

Although the present invention has been described in connection with a preferred embodiment, it should be understood that modifications, alterations, and additions can be made to the invention without departing from the scope of the invention as defined by the claims.

What is claimed is:

1. A method for neutralizing a target pathogen or substance, comprising the steps of:
exposing a volume of water or air to a filtration device, the filtration device comprising: a filtration medium comprising a plurality of microcapsules, each of said microcapsules containing a neutralizing agent, wherein said neutralizing agent is released from at least one of said plurality of microcapsules in response to an activation signal;
detecting the target pathogen or substance;
initiating an activation signal upon detection of the target pathogen or substance;
releasing the neutralizing agent from at least one of said plurality of microcapsules in response to the activation signal; and
neutralizing the target pathogen or substance with the released neutralizing agent.

2. The method of claim 1, wherein said filtration medium is permeable to air.

3. The method of claim 1, wherein said filtration medium is permeable to water.

4. The method of claim 1, wherein the microcapsules comprise two or more different neutralizing agents.

5. The method of claim 4, wherein a plurality of the microcapsules comprise a first neutralizing agent, and a different plurality of the microcapsules comprise a second neutralizing agent.

6. The method of claim 1, wherein said microcapsules burst or dissolve in response to said activation signal.

7. The method of claim 1, further comprising the step of releasing the resulting neutralized volume of water or air from the filtration device.

8. The method of claim 1, wherein the filtration device comprises a second filtration medium, said second filtration medium adapted to filter said neutralized target pathogen or substance from said volume of water or air.

9. A system for neutralizing a target pathogen or substance, comprising:
a filtration device, the filtration device comprising a filtration medium comprising a plurality of microcapsules, each of said microcapsules containing a neutralizing agent; and
a sensor adapted to detect the target pathogen or substance and, upon detection of the target pathogen or substance, send an activation signal to the plurality of microcapsules;
wherein said at least one of said plurality of microcapsules is adapted to release said neutralizing agent in response to the activation signal.

10. The system of claim 9, wherein said filtration medium is permeable to air.

11. The system of claim 9, wherein said filtration medium is permeable to water.

12. The system of claim 9, wherein the microcapsules comprise two or more different neutralizing agents.

13. The system of claim 9, wherein a plurality of the microcapsules comprise a first neutralizing agent, and a different plurality of the microcapsules comprise a second neutralizing agent.

14. The system of claim 9, wherein said microcapsules burst or dissolve in response to said activation signal.

15. The system of claim 9, wherein the filtration device further comprises a second filtration medium, said second filtration medium adapted to filter said neutralized target pathogen or substance from said volume of water or air.

16. A method for neutralizing a target pathogen or substance using a neutralizing agent, comprising the steps of:
exposing a volume of water or air to a filtration device, the filtration device comprising: (i) a sensor adapted to detect the target pathogen or substance; and (ii) a filtration medium comprising a plurality of microcapsules, wherein a first group of said plurality of microcapsules comprise a first neutralizing agent precursor and a second group of said plurality of microcapsules comprise a second neutralizing agent precursor, and further wherein said first and second neutralizing agent precursors are released from said plurality of microcapsules in response to an activation signal;
detecting, by said sensor, the target pathogen or substance;
initiating an activation signal upon detection of the target pathogen or substance;
releasing the first and second neutralizing agent precursors from said plurality of microcapsules in response to the activation signal, wherein the released first and second neutralizing agent precursors interact to create the neutralizing agent;
neutralizing the target pathogen or substance with the neutralizing agent; and
releasing the resulting neutralized volume of water or air from the filtration device.

17. The system of claim 16, wherein said filtration medium is permeable to air.

18. The system of claim 16, wherein said filtration medium is permeable to water.
